(19) United States
(12) Patent Application Publication
(10) Pub. No.: US 2013/0345703 A1
(43) Pub. Date: Dec. 26, 2013

(54) METHOD FOR MANIPULATING OBJECTS EMPLOYING NANOTECHNOLOGY

(71) Applicants: David Y. Phelps, Louisville, KY (US); Vasiliy Abramov, Louisville, KY (US)

(72) Inventors: David Y. Phelps, Louisville, KY (US); Vasiliy Abramov, Louisville, KY (US)

(73) Assignee: Creosalus, Inc., Louisville, KY (US)

(21) Appl. No.: 13/955,577

(22) Filed: Jul. 31, 2013

Related U.S. Application Data
(63) Continuation of application No. 12/471,421, filed on May 25, 2009.

(51) Int. Cl. B25J 11/00; A61B 18/18

(52) U.S. Cl. CPC B25J 11/00; A61B 18/18
USPC 606/45; 414/800; 606/49

(57) ABSTRACT

A method for capturing small objects in a confined space such as an artery or duct in a body by bringing an elongated flexible body having a distally disposed operative head provided with a plurality of nanostructures into proximity with the object. When in proximity intermolecular forces are created between the nanostructures and the object that attract the object to the operative head. By application of high frequency electrical current the nanostructures act as electrical knives to cut away or cauterize an object.

### RELATED APPLICATION

This application is a continuation of application Ser. No. 12/471,421, filed May 25, 2009, entitled DEVICE FOR MANIPULATING OBJECTS EMPLOYING NANOTECHNOLOGY.

### FIELD

This invention relates to nanotechnology and to methods employing nanotechnology for manipulating objects in a confined space and more particularly to methods for securing and removing objects from within a body.

### BACKGROUND

Surgical gripping instruments are well known in the art for use in entering body cavities for removal of tissue, clots, stones and the like. These devices rely on loops or grasping arms that are manipulated by the surgeon to grasp an object for removal from the cavity. Such cavities may include vessels as are found in the vascular system or the non-vascular system. Additionally, this device may be used as a grasper in laparoscopic or arthroscopic surgery.

An exemplary structure of such surgical gripping instruments comprises a manually operated device associated with a transmission cable having cable sheathing and a cable core. Gripping elements are coupled to the end of the cable core on the patient's side. The gripping elements have connection parts controlled by the end of the cable sheathing on the patient's side which is movable relative to the cable core or by an end sleeve connected with the operating end of the cable sheathing. The end of the cable sheathing is connected with a finger hold of the manual operating device and the cable core connected with a thumb hold of the manual operating device. The thumb and finger holds are movable relative to each other to effect relative motion of the cable sheathing and cable core. Such a surgical gripping instrument is used especially for the purposes of bronchoscopy, bulboscopy, coloscopy, duodenoscopy, endoscopy and gastroscopy.

These prior art devices employ opposed active gripping arms, snares or coils which must be actuated such as by moving opposed arms together or by twisting or maneuvering the device in the case of coils and snares to secure an object being maneuvered. It is generally necessary that the grasping portion of the device be moved to the distal portion of the object being grasped so that the grasping arms, snare or loop achieves a purchase on the object for maneuvering. Oftentimes, as mentioned, in the case of snares or coils, the operative head of the device is rotated to wrap the snare or coil around the object. Once the object is grasped the operative head is maneuvered with the object secured thereto.

In surgical catheter type instruments for insertion in a body for removing or otherwise modifying an object in a confined space such as a vein, duct or the like means are normally provided for extending and withdrawing the operative or distal head of the device. Such means broadly includes a housing in which an axially movable member is connected to the sheathing of a flexible cable that carries the operative head on its distal end. A thumb hold is connected with the end of the cable sheathing on the operating or proximal side and an index finger-middle finger hold is connected with the end of the cable core on the operating side. If the handling physician or the surgical nurse bring the index finger-middle finger hold closer to the thumb hold, the core is retracted relative to the cable sheathing on the patient's side and the connection parts of the gripping elements move into the cable sheathing or the end sleeve with the consequence that the gripping elements are closed in a tong-like manner. Likewise tissue specimens can be gripped and taken.

For example, U.S. Pat. No. 5,084,054 teaches a surgical gripping instrument that includes a support assembly, a slide mounted on the support assembly for relative movement with respect thereto, a wire movable with the slide relative to the support assembly, gripping means at the distal end of the wire, and a sheath enveloping the wire. Movement of the wire a predetermined distance relative to the sheath causes the gripping means to be actuated. A means are provided to enable the slide to be moved a distance relative to the support assembly less than the predetermined distance causing sufficient displacement of said sheath relative to the gripping means to actuate the latter.

U.S. Pat. No. 4,178,810 teaches an apparatus for manipulating a medical instrument such as an endoscopic device that comprises a plunger slidably disposed within a slotted support tube and movable via a finger sleeve surrounding the tube and coupled to the plunger. A control wire extends through an inner coil and a surrounding outer coil; the adjoining ends of the coils within the support tube are brazed or welded together, and the other end of the outer coil is secured to the end of the support tube by set screws. An end stop is attached to the end of the control wire and is normally seated against one end of a cavity within the plunger by a spring. To close the forceps cup to sever and extract the desired tissue sample, a finger sleeve and plunger are axially moved whereby the end stop seats at its one end of the cavity and draws the control wire through the inner coil.

Canadian patent application 2,551,191 teaches microfabricated MEMS devices that comprise electrothermally-driven opposed microgrippers with integrated dual-axis force sensing capabilities. The microgrippers act as jaws and the gripping motion is produced by an actuator, such as a bent-beam actuator. Such devices are subject to the same disadvantages as more conventional gripping devices in that it is necessary to at least partially surround the object being removed in order that the microgrippers can obtain a purchase on the object.

The following disadvantages have been recognized in the surgical gripping instruments art. All previous devices must go to a distal portion of the object to be removed. They rely on the skill of the physician in manipulating the snare or arms of the operative head so that it “lassoes” or grasps the offending article. If the article is too large or irregular it could block the device from getting to the distal end portion of the object and preclude obtaining a purchase of the article sufficient to remove the article. A danger is that the article may be forced further upstream or into the wall of the vessel as one tries to fish the instrument past the article.

### SUMMARY OF THE INVENTION

The present invention relates to a method for maneuvering small objects utilizing nanotechnology to secure an object and for conducting a procedure on the object such as, for example, removing a clot or stone from an artery or duct in a body or for removing blockage from a valve or line in a machine or instrument. In accordance with the invention an object is secured without actuating moving parts or otherwise manipulating the device to secure the object.

In accordance with one aspect of the invention a method is defined wherein an elongated flexible body carrying on its distal end a operative head provided at least on its distal surface with a plurality of nanostructures is inserted within a confined space such as a body cavity or an artery, vein or duct containing an object to be removed such as a clot or stone. The operative head is brought into proximity with a surface of the object that faces the operative head until there an attractive force is formed between the nanostructures on the operative head and the surface of the object facing the operative head. Because of the nanoscale dimensions, intermolecular and Van der Waals forces are created between the nanostructures and the object when they are in proximity to one another. These intermolecular forces are capable of capturing an object such as a clot, small stone or individual cells. The elongated flexible body can then be withdrawn bringing out the object secured by the operative head

The nanostructures comprise silicon or carbon structures in the form of tubes or fibers. In particular the carbon structures can be as thin as a one-atom thick graphene sheet of graphite rolled up into a fiber or cylinder to define a nanoscale fiber or tube with the diameter on the order of about one nanometer. The nanoscale tubes can also comprise multi wall carbon structures.

In one embodiment of the method nanofibers are embedded at one end in a suitable substrate and oriented essentially vertically on the substrate with the extending distal ends of the nanofibers disposed essentially in the same plane as the plane of the substrate to define an adherent retrieval surface.

In yet another embodiment the distal portions of the nanofibers are bent so that their distal portions lie essentially parallel to the distal surface of the core to provide maximum surface area contact between the nanofibers and the object.

In still yet another embodiment the nanofibers are biased upwardly outwardly or upwardly inwardly with respect to the plane of the distal surface of the core and in some embodiments a single substrate carries a pattern where portions of the fibers are biased upwardly outwardly and other portions where the fibers are biased upwardly inwardly to provide an even greater securing force on an object.

In accordance with the method of the invention the object is secured without the necessity of surrounding and reaching the distal portion of the object. In this manner the danger of forcing an object further into an artery or duct or breaking the object into smaller pieces that may circulate through the vessel or duct with possible dire results in an attempt to grasp it is greatly minimized.

In another embodiment of the invention, means may be incorporated in the method for determining the relative position of the operative head and the object. Such means may be, for example, either visual such as an optical microscope or an electron microscope or by an electronic force feedback system.

Other advantages of the invention will be seen from the following description of the invention taken with the drawings in which:

### BRIEF DESCRIPTION OF THE DRAWINGS

FIG. 1 is side view of a device in accordance with the method of the invention broken away for compactness of illustration;

FIG. 2 is a side view of the distal end of the device of FIG. 1 in enlarged scale and partially broken away for compactness of illustration showing the device with its operative head secured to a facing surface of an object in a duct of a body;

FIG. 3 is a side elevation of another embodiment of a surgical device in accordance with the invention including a fiber optic line for visual feedback during operation; and

FIG. 4 is a side view of the manipulative head of the device illustrating another orientation of the nanofibers.

FIG. 5 is a side view of the operative end illustrating the nanofibers bent at the distal end portion.

### DESCRIPTION OF THE PREFERRED EMBODIMENTS

As used herein the term “nanoscale” is used to refer to components of 100 nanometers or less. The terms “nanostructure”, “nanotube”, “nanofiber”, “nanoscale tubes” and “nanoscale fibers” are used interchangeably and define thread-like tubular or solid fibrous structures having a diameter ranging from about 0.1 nm to about 100 nm.

The invention will be described in connection with a method of use in a human or animal body such as for the removal of an object from a blood vessel, duct. However, it will be understood that the present invention is highly suited for clearing blockages by small objects in instrument lines, valve nozzles and the like where small objects can block the proper functioning of the instrument or valve. Such blockages are often difficult to remove because of limited access to the blockage and because of the normally confined space and the size of the blockage its removal becomes very difficult. In some cases when attempting to grasp the object it is broken and pieces remain that can interfere with the operation or damage sensitive instrument components.

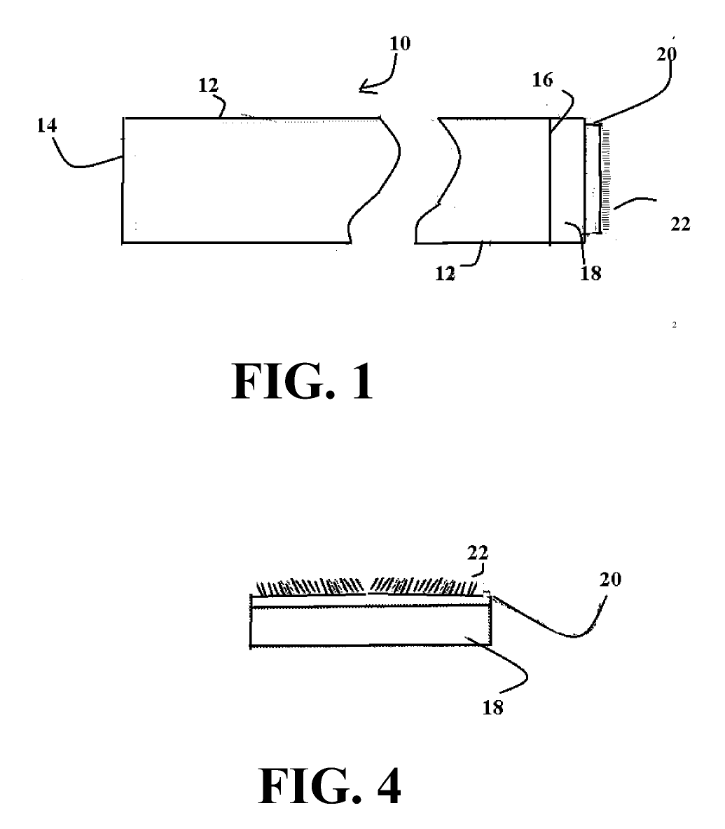

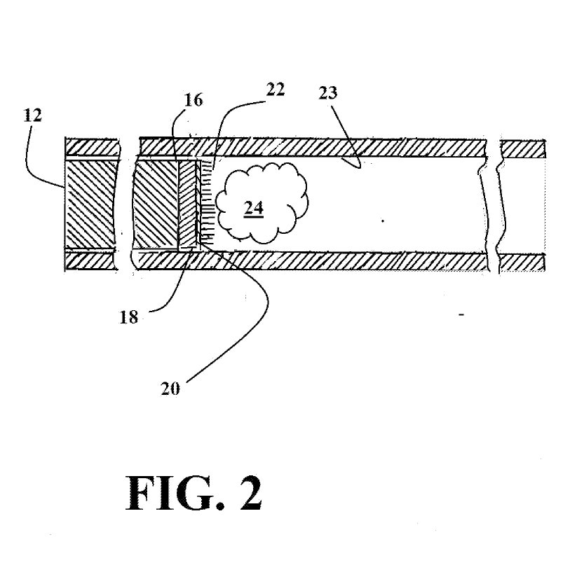

Referring to FIG. 1 and FIG. 2 there is illustrated a device 10 for carrying out the method of the invention and illustrating the device in proximity to an object. The device 10 comprises a flexible core 12 of suitable dimensions for insertion into a confined space within the human body and for capturing and removing objects from the confined space. The confined space may be variously a vessel, open region such an aneurysm, or a duct. The object to be removed may include calcified objects, clots and cells. It is immaterial that the object to be removed is covered with blood or other fluids.

In general, the core 12 is produced in a manner similar to devices used in endovascular catheterization procedures. The core 12 comprises a proximal end 14 and a distal end 16. A conventional finger hold (not shown) is attached to the proximal end 14 of the core 12 to manipulate the device 10. In general, the core 12 may be of stainless steel acceptable for use in intravascular devices or may be of any other material which is known to be safe and efficacious in such practice. One common material is a series of nickel-titanium alloys, some of which are known as nitinol in the art.

An operative head 18 is defined at the distal end 16 of the core 12. The operative head 18 includes a substrate 20 in which are embedded a plurality of nanoscale fibers or tubes 22. As illustrated the nanofibers 22 are embedded in the substrate 20 at one end and extend normal to the plane of the substrate with the distal ends of the nanofibers lying essentially in a plane parallel to the plane of the substrate to define a gripping surface of the operative head 18.

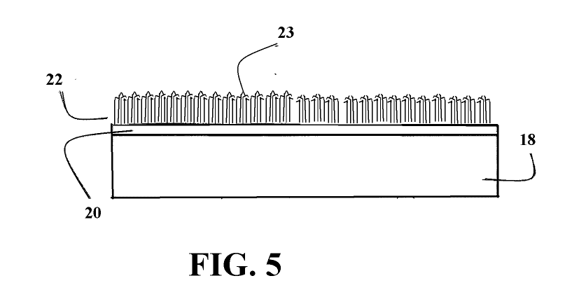

Depending on the procedure for which the device 10 is to be used it may be desireable to have the proximal ends of the nanofibers 22 lie essentially parallel to the plane of the of the substrate 20 in order to present more surface area to an object being manipulated (FIG. 5). Also, the nanofibers 22 may be arrange so that they are biased upwardly to the axis of the core 12. As illustrated by FIG. 4 the nanofibers 22 are biased away from the axis of the core 12. If maximum adhesion is unnecessary the nanofibers 22 can be randomly disposed on the substrate.

Preferably the nanofibers 22 are carbon fibers that are utilized for their high performance and mechanical properties. Carbon nanotubes for example can be made of thin, narrow sheets of graphene graphite that roll up and clump together to form nanotubes. The nanotubes may be single wall or double wall. However, silicon can be also used as the structural material for the nanofibers 22. Carbon nanostructures are physically more robust than silicon nanofibers and for this reason are preferred. Since the device 10 utilizes weak Van der Waals forces that at nanoscale are strong enough to create an attraction between the fibers 22 and an object and because ordinarily little or no physical stress will be applied to the nanofibers 22, either silicon or carbon fibers may be used with equal results.

The substrate 20 may be deposited on the distal end 16 of the core 12 by procedures well known in the art such as vapor deposition. The substrate may comprise SiO₂ or a polymer. Methods for depositing carbon nanotubes and nanofibers on a substrate in a desired orientation have been developed and are known in the art. For example, nanotubes can be deposited on a prepolymer substrate that is subsequently cross-linked to trap the ends of the fibers in the polymer substrate. An alternative method is to grow the nanofibers on the substrate by plasma enhanced chemical deposition. The particular methods for forming, anchoring and orienting such nanofibers are known in the art and such methods are not per se part of the present invention.

Silicon nanofibers can be formed by vapor deposition of a silicon layer over a silica layer followed by the application of a suitable photoresist. The silicon uncovered by the photoresist is subjected to etching while the covered portions, following removal of the photoresist, form the silicon nanofibers 22. The nanofibers 22 may also be formed on the substrate 20 after it has been deposited on the core 12 or alternatively the fibers can be formed on substrate sub assemblies which are thereafter secured on the end of the core.

The shortest dimension (OD) of a nanotube or nanofiber is, by definition, in the nanometer range and is typically from about 0.1 nm to about 100 nm and most often from about 2 nm to about 50 nm. Typical lengths can range from about 20 nm to about 100 μm. Often, the aspect ratio, that is the ratio of the length to the diameter, of carbon nanotubes is at least 10,000, though this is not required. This being said, it is noted that physical dimensions of nanostructures can vary considerably.

In accordance with the method of the invention the core 12 of the surgical device 10 is inserted in a body opening 23, such as a body cavity, vessel or duct. As the nanofibers 22 come into proximity of an object 24 in the vessel or duct, intermolecular forces such as Van der Waals forces begin to exert an attraction between the nanofibers and the object to be removed. Continuing to move the nanofibers 22 toward the object to create sufficient intermolecular attractive fore to capture the object. Because of the nanodimensions of the numerous nanofibers 22 on the operative head 18, the total attractive force between the nanofibers and the facing surface of the object 24 is substantial and the object is captured. The object is then removed from the vessel or duct as the core 12 is withdrawn. In this fashion the device operates without moving parts and it is unnecessary that components of the device 10 be activated or the device maneuvered to secure the object nor is it required that the operative retrieval portion of the device 10 surround the object to be withdrawn or otherwise reach its distal portion in order grasp or lasso the object. The danger of forcing the object further into the vessel or duct or breaking the object into smaller pieces that may circulate through the vessel or duct with possible dire results is avoided. The action of the nanofibers 22 is unaffected by the surface condition of the object 24 and it is immaterial that the surface of the object is covered with fluids. In addition the effectiveness of the nanofibers does not diminish with use unless they become disoriented or are otherwise damaged.

In surgical procedures employing the method of the invention it is highly desirable to be able to determine the proximity of the operative head 18 to the object 24. Among other things some or all of the nanofibers 22 can possibly be damaged by excessive contact force with the object 24. Also, excessive contact force may result in forcing the object 24 further into the vessel or duct 23.

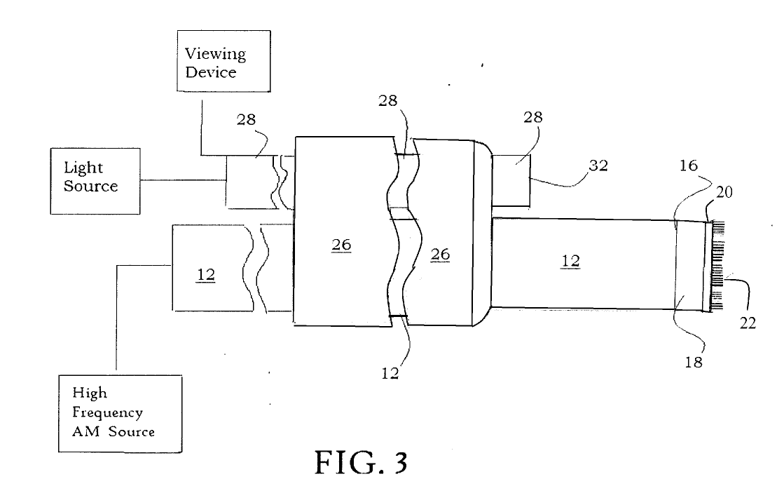

As shown in FIG. 3 the device 10 employed in the method comprises an outer tubular sheath 26 in which is disposed the core 12 for longitudinal movement therein. The proximal end of the core 12 includes a suitable conventional finger control (not shown) for moving the core longitudinally so that the distal end 16 and operative head 18 can extend beyond the distal end of the sheath for contact with the facing surface of an object to be removed. The operative head 18 of the core 12 comprises a suitable polymer substrate 20 in which are vertically embedded carbon or silicon nanofibers 22. An optical fiber 28 that defines a proximal end 30 and a distal end 32 extends longitudinally through the sheath 26. The distal end 32 of the optical fiber 28 terminates at the distal end of the sheath and the proximal end 30 is in optical communication with a radiation source and a viewing device such as an optical microscope or an electron microscope and the radiation source may emit visible light or infrared radiation. As the operative head approaches an object in a vessel or duct the reflected radiation can be observed by the viewing device to determine the proximity of the operative head 18 to an object or by sensing a change in the reflected radiation.

Although the device 10 has been described in connection with an optical or radiation detection system to determine proximity of the operative head 18 to the object it will be understood that the technology in this field is rapidly changing. Thus other systems such as force field systems can be employed to determine proximity of the head 18 to the object.

Although the method of the invention has been described in connection with retrieval of objects from a confined space, nanofibers and nanotubes have other highly beneficial properties that render the method of this invention useful for other procedures. For example, the method will find use in electro cauterization for removal of objects, such as polyps and other growths, from a body. In this embodiment advantage is taken of the excellent electrical properties of the nanofibers 22. For this embodiment the device 10 of FIG. 1 is in electrical communication with a source of high frequency alternating current. The core 12 is electrically insulated and is composed of an electrically conductive material such as stainless steel for conducting the current to the carbon nanofibers 22. A second electrode is applied to the skin near the surgery site. This electrode collects the electricity from the body and completes the circuit. A grounding pad may be placed on the person's body (usually the thigh) before the surgery to protect the patient from burns although at nanoscale the flow of current is small and the danger of injury to the patient is low. When the Nano fibers are in contact with the object, the high frequency current is activated resulting in converting the nanofibers into a plurality of nanoscale electrosurgical knives. The current passing through the tissue cuts the tissue away. When the surgery is complete any remaining portion of the now separated tissue adheres to the ends of the nanofibers and is retrieved as the operative head 18 is withdrawn from the body opening 23.

In accordance with the invention there is provided method for retrieval of an object from a confined space that avoids the problems associated with prior art gripping/grasping methods and devices including prior art nanoscale grasping devices. In general these prior art methods employ devices comprising grasping means that must approach the distal portion of the object. This requires that the object be at least partially surrounded by the grasping means in order that the grasping means can obtain a purchase on the object to be removed. In accordance with the method of the invention it is necessary only to contact the facing surface of an object and by taking advantage of intermolecular attraction forces that at nanoscale are strong. The necessity of surrounding the object to grasp it, which can result in damaging the object or forcing it or pieces of it further into a vessel or duct is avoided.

While the method and the nanoscale devices employed therewith disclosed herein have been particularly shown and described with reference to the exemplary embodiments thereof, those of ordinary skill in the art will understand that various changes may be made in the form and details herein without departing from the spirit and scope of the disclosure and the appended claims. Those of ordinary skill in the art will recognize or be able to ascertain many equivalents to the exemplary embodiments described specifically herein by using no more than routine experimentation. Such equivalents are intended to be encompassed by the scope of the present disclosure and the appended claims.

1. A method for manipulating objects in a confined space employing nanotechnology comprising
a. inserting into said confined space an elongated flexible body including a distal end defining an operative head having disposed thereon a plurality of nanostructures,
b. moving said flexible body in said confined space to bring said operative head into proximity of a surface of said object to cause intermolecular forces to begin to exert attraction between said nanostructures and said object,
c. continuing to move said operative head toward said object to generate sufficient attractive force for capture of said object by said operative head,
d. withdrawing said elongated flexible body and said captured object from said confined space.

2. The method of claim 1 wherein said object defines a surface area facing said operative head and said intermolecular force is established between said nanostructures and said facing surface.

3. The method of claim 1 wherein said nanostructures are selected from the group consisting of carbon nanofibers and carbon nanotubes and combinations thereof.

4. The method of claim 1 wherein said nanostructures are selected from the group consisting of silicon nanofibers and silicon nanotubes and combinations thereof.

5. The method of claim 3 wherein said nanostructures are oriented to extend normally from the operative head.

6. The method of claim 3 wherein said nanostructures are randomly oriented on said operative head.

7. The method of claim 4 wherein said nanostructures are oriented to extend normally from the operative head.

8. The method of claim 4 wherein said nanostructures are randomly oriented on said operative head.

9. The method of claim 1 further including apparatus for determining the proximity of said operative head to said object.

10. The method of claim 9 wherein the feedback apparatus comprises an optical fiber that terminates at said operative head, said optical fiber being in communication with a source of radiation and a viewing device.

11. A method for manipulating an object employing nanotechnology comprising the steps of:
a. inserting into a body opening an insulated, electrically conductive, elongated flexible member having a distal end defining an operative head having disposed thereon a plurality of carbon nanostructures;
b. electrically connecting said member to a source of high frequency electrical current;
c. moving said operative head into electrical contact with said object;
d. applying an external electrode on the surface of the body adjacent the cauterization site thereby to complete an electrical circuit; and
e. activating the high frequency electrical current to cauterize or cut away said object; and
f. capturing at least portions of said object on said nanostructures by intermolecular force created between said nanostructures and said portions: and
g. withdrawing said elongated flexible body and said captured portions from said body opening.

12. The method of claim 11 further including placing a grounding pad on the surface of said body.

13. The method of claim 11 wherein said carbon nanostructures are selected from the group consisting of carbon nanotubes and carbon nanofibers and combinations thereof.
